(19) United States
(12) Patent Application Publication
(10) Pub. No.: US 2025/0210172 A1
(43) Pub. Date: Jun. 26, 2025

(54) ENHANCED PRESCRIPTION MANAGEMENT SYSTEM

(71) Applicant: PRESCRYPTIVE HEALTH, INC., Redmond, WA (US)

(72) Inventors: Christopher Scott Blackley, Redmond, WA (US); Kevin C. Young, Redmond, WA (US); Hani Rachidi, Redmond, WA (US); David Alexaender Smith, Redmond, WA (US)

(21) Appl. No.: 19/074,053

(22) Filed: Mar. 7, 2025

Related U.S. Application Data
(63) Continuation of application No. 16/827,218, filed on Mar. 23, 2020, now abandoned.
Application No. 16/827,218 is a continuation-in-part of application No. 16/177,314, filed on Oct. 31, 2018.

(51) Int. Cl. G16H 20/10; A61B 5/00; G16H 10/60; G16H 40/67; G16H 80/00

(52) U.S. Cl. CPC G16H 20/10; A61B 5/74; G16H 10/60; G16H 40/67; G16H 80/00

(57) ABSTRACT

A system for providing to a patient a user experience for securely submitting a prescription to a pharmacy is provided. The system receives a notification of a prescription prescribed by a prescriber for the patient. The system displays to the patient information on drug options relating to the prescribed drug. The system then receives from the patient a selection of a drug option. The system displays to the patient pharmacy options relating to the selected drug option. Each pharmacy option identifies a pharmacy. The system receives from the patient a selection of a pharmacy option. The system then directs that an indication of the prescription be sent to the pharmacy identified by the selected pharmacy option so that that pharmacy can dispense the prescription.

### CROSS-REFERENCE TO RELATED APPLICATION

This application is a continuation of U.S. patent application Ser. No. 16/827,218 filed on Mar. 23, 2020, which is a continuation-in-part of U.S. patent application Ser. No. 16/177,314 filed on Oct. 31, 2018, which are hereby incorporated by reference in their entirety.

### BACKGROUND

Current computerized technologies relating to the prescription process, that is prescribing of drugs and the dispensing or filling of prescriptions, are both inefficient and costly. When a prescriber (e.g., physician) writes a prescription, the prescriber submits the prescription into a prescriber computer system of the prescriber. The prescription includes an identification of the patient, name of the drug, dosage, quantity, refill information, pharmacy to dispense, and so on. The prescriber computer system transmits the prescription to a prescription routing service. Upon receiving the prescription, the prescription routing service checks whether the identified pharmacy is registered with the routing service. If so, the prescription routing service transmits the prescription to the pharmacy. If the pharmacy has access to information on the prescription coverage (if any) of the patient, the pharmacy determines whether the prescribed drug is covered and, if so, the covered price amount. The pharmacy prepares the prescription to be dispensed. When the patient arrives at the pharmacy, the patient pays that patient prices, which is the difference (if any) between the pharmacy price that the pharmacy charge and the covered price amount.

A patient may be a member of a prescription benefit plan that is provided by a plan sponsor such as the patient's employer, a governmental entity, and so on, who may pay a portion of the prescription drug cost as defined under the plan. To help control costs and improve medical outcomes, such a plan sponsor may define a formulary, which lists the medicines covered by the plan. A formulary may specify the medicines and dosages that are approved for the treatment of each disease (or, more generally, condition), including “off-label” uses. A formulary may also specify, for a disease, the order in which medicines are approved to be paid for by the sponsor under the plan. For example, the formulary may specify that drug A needs to be prescribed before drug B can be paid for under the plan. Because the creating and maintaining of a formulary can be expensive, sponsors often rely on pharmacy benefit managers (“PBMs”) that provide computer systems to manage formularies, to pay pharmacies, and to receive payment from sponsors.

The current prescription process has several disadvantages. First, if a prescribed drug is not covered by the prescription coverage (e.g., not in formulary), the patient may not find out about the lack of coverage until arriving at the pharmacy to pick up the prescribed drug. After the patient finds out about the lack of coverage, the patient may need to contact the prescriber to discuss alternative drugs. A similar process to handling the original prescription is then repeated. Again, the patient may not find out whether the alternative drug is covered until arriving at the pharmacy. Second, although a prescriber may provide a patient with a ballpark price of a prescribed drug, the patient typically will not find out the patient price until arriving at the pharmacy. The patient price can vary based on the covered price amount and the pharmacy price. The pharmacy price, and thus the patient price, can vary because of the purchasing power of the pharmacy, the desire to increase foot traffic for purchasing non-prescription items, the desire to dispense a prescription originally in hopes that the patient will return for refills, the rebates offered by drug manufacturers, and so on. If the patient price is more than the patient is willing to pay (or can afford), then the patient may need to contact the prescriber to discuss alternative drugs. Third, a pharmacy may not have the prescribed drug in inventory. The prescribed drug may be in short supply because of demand or manufacturing problems. Also, the prescribed drug may have been subject to a recent recall and is no longer available. If the prescribed drug is not available, then the patient may need to contact the prescriber to discuss alternatives. A prescribed drug may also require prior authorization from the plan sponsor before it is dispensed to be covered, and therefore paid for under the plan. A patient would not find out about prior authorization until arriving at the pharmacy, requiring the prescriber to follow up with the plan administrator to obtain approval for the patient. It is both costly and inefficient for a prescriber to deal with a patient a second time and rewrite a prescription and a pharmacy to deal with prescriptions that a patient cannot pay for or when the prescribed drug is not available. Fourth, many drugs are limited distribution, such as specialty drugs, available only at specialty pharmacies. The prescriber would not be aware that the local pharmacy would not carry the drug, and the patient would not find out until arriving at the pharmacy.

Some services are available that provide pricing information to patients. For example, some services provide an application and a web site for comparing pharmacy prices. To use such a service, a patient enters the name of the prescribed drug, dosage, and quantity. The service looks up the pharmacy prices of various pharmacies (e.g., nearby the patient) and displays the results. There is, however, no guarantee that a pharmacy will honor the prescription price. For example, the drug manufacturer may have recently increased the price of or may no longer provide rebates for the prescribed drug, the pharmacy may no longer offer discounts for the prescribed drug, and so on. Due to the inherent complexities of the current prescription pricing and distribution system, many such services present the patient with an “estimated price” which will be different from, and often greater than, the actual price. Therefore, these services often do not result in providing patients with the information needed to make accurate, informed choices when seeking the most affordable medications they need. Further, the prices shown in such services are the cash price, not a personalized patient price as calculated by the distinct plan/pharmacy combination and the patient's current deductible status.

### BRIEF DESCRIPTION OF THE DRAWINGS

FIGS. 1-3 illustrate a user experience for a patient that is provided by the EPP system in some embodiments.

FIG. 4 is a block diagram that illustrates components of the EPP system in some embodiments.

FIG. 5 includes flow diagrams that illustrate the processing of an EPP server and EPP client in some embodiments.

FIG. 6 is a flow diagram that illustrates the conducting of a pharmacy auction.

FIG. 7 is a diagram that illustrates the flow of data relating to prescription options.

FIG. 8 is a flow diagram that illustrates the processing of a claim by a pharmacy benefit manager system in some embodiments.

FIG. 9 is a flow diagram that illustrates processing of a process patient response component of a pharmacy benefit manager in some embodiments.

### DETAILED DESCRIPTION

A method and system for providing to a patient an enhanced user experience for controlling the prescription process are provided. In some embodiments, an enhanced prescription process (“EPP”) system provides information to a patient in real-time to assist the patient to determine whether a drug is covered, identify alternative drugs, determine actual patient prices that will be honored by pharmacies and when the prescribed drug would be ready for pickup, select a pharmacy to dispense the prescription, and so on. The EPP system may also provide to the patient a confirmation of receipt of the prescription by the pharmacy. Once the prescriber submits the prescription electronically, the information may be provided in real-time in the sense that the information is provided to the patient while with the prescriber (e.g., at a doctor's appointment) at the time the prescription is written so that the patient can ask the prescriber questions about alternative drugs and that the most appropriate prescription can be sent to a pharmacy based on information provided to the patient by the EPP system.

In some embodiments, the EPP system includes an EPP server and an EPP client. The EPP server may be under control of an “EPP pharmacy” registered with a prescription routing service. The EPP pharmacy may also be registered with governmental authorities and dispense some prescriptions and transfer other prescriptions to other pharmacies. Alternatively, the EPP pharmacy may not dispense any prescriptions. Rather, the EPP pharmacy may only receive prescriptions and transfer them to pharmacies selected by patients. The EPP server may provide a registration service for registering patients and interface with systems such as PBM systems and pharmacy systems. The registration service may allow patients to directly register and sponsors to register its participants with the EPP system. The EPP system interfaces with the PBM systems to determine drug options such as drug coverage, alternative drugs, covered prices, and so on as specified in part by formularies. The EPP system interfaces with pharmacy systems to determine patient prices (and any incentives), determine drug availability, submit prescriptions, confirm receipt of prescriptions, and so on. The EPP server communicates with an EPP client to send notifications of prescriptions, drug options, pharmacy options, pricing, and pharmacy confirmations and receive pharmacy options.

The EPP client may be provided as an EPP application or via an EPP web site. The EPP application may be installed on a portable computer such as a smartphone, tablet, or laptop. The EPP web site may be accessible via any computer with a web browser. When the EPP application is installed on a portable computer, the patient may bring the portable computer to a visit with the prescriber so that prescription can be finalized in real-time that is during the visit. The prescriber may make available to the patient a computer with a web browser, so the patient can access the EPP web site and the prescription can be finalized even if the patient does not have the EPP application. Alternatively, the patient may use the EPP application or the EPP web site after a prescriber visit to finalize a prescription. However, the patient may need to contact the prescriber (e.g., via a call or an email) to discuss and possibly modify the prescription. The EPP system may interface with systems of the prescriber to facilitate the discussion. For example, the EPP system may automatically log in to a secure system of the prescriber to send and receive messages. Alternatively, or additionally, the patient may need to contact the pharmacist dispensing their medication, or vice versa, so that the pharmacy may provide patient consultation regarding, among other things, safe and appropriate use of the medication before dispensing the medication. The EPP system may interface with systems of the pharmacy to facilitate the consultation.

In some embodiments, an EPP client executing on a device of a patient may receive a notification of a prescription prescribed by a prescriber during a visit. The notification may be sent by the EPP server in response to receiving a prescription from a prescription routing service, which is a prescription intermediary that receives the prescription electronically from the prescriber. The notification (or content accessed after the notification is received) may include an identification of the prescribed drug, dosage, and quantity. The notification may also include drug options relating to the prescription such as alternative drugs, prior authorization, and pricing information. The EPP client displays the drug options to the patient. The patient may discuss the drug options with the prescriber. If an alternative drug is selected, the prescriber electronically submits a prescription for the alternative drug. An alternative drug may be selected before or after any prescription is entered into the prescription system. If selected before, the EPP client may send possible prescription information (e.g., drug, dosage, and quantity) to the EPP server, which can then provide a notification with the appropriate drug options. The EPP client may allow a patient to enter manually (e.g., prior to the prescription being written) possible prescription information that is sent directly to the EPP server. The patient may also send an image of the prescription or possible prescription information to the EPP server for optical character recognition. Whether the EPP server receives a prescription from the prescription routing service or receives possible prescription information from an EPP client, the EPP server identifies pharmacy options and sends them to the EPP client. The EPP client displays the pharmacy options. Each pharmacy option identifies a pharmacy and may include pricing information. The EPP server may start collecting pharmacy pricing information when a prescription or possible prescription information is received. The patient then selects a pharmacy, and the EPP client sends to the EPP server an indication of the selected pharmacy. The EPP server then sends the prescription to the selected pharmacy. In some embodiments the EPP server may send the prescription to the selected pharmacy via a prescription routing service, which then forwards the prescription to the selected pharmacy. Alternatively, the EPP server may interface directly with pharmacy systems to send the prescription to the selected pharmacy. When the EPP server receives confirmation from the pharmacy and notifies the EPP application, the EPP application displays confirmation information. The confirmation information may include a Quick Response (“QR”) code that can be used to identify the prescription when the patient arrives at the pharmacy, collect any incentives (e.g., 10% off in-store purchases) offered to the patient, and so on.

In some embodiments, a prescription intermediary may be a service that receives the prescription directly from the prescriber and forwards prescriptions to the pharmacy identified by the prescriber. Alternatively, the prescription intermediary is a service that receives the prescription via a prescription routing service, which receives the prescription directly from the prescriber and distributes the prescription to the prescription intermediary. In such a case, the prescription intermediary may be a service that is controlled by an EPP pharmacy and that transfers or forwards the prescription to the selected pharmacy.

In some embodiments, the EPP server may collect pharmacy information from pharmacy in advance of receiving a prescription and store the pharmacy information for retrieval when a prescription is received. The EPP server may also collect pharmacy information in real-time after a prescription is received. In such as case, the EPP server may send prescription information to various pharmacies, such as those previously selected by a patient, geographically near a patient, approved by a sponsor, and so on, and receive pharmacy information in return. The EPP server may also conduct an auction in which pharmacies bid to dispense a prescription. For example, after receiving a prescription, the EPP server may send bid requests that identify the prescription and may include information relating to plan coverage. The pharmacies then send bid responses. A bid response may include the patient price and incentives, discounts on refills, loyal program benefits, and so on. The incentives may be provided by manufacturers (e.g., mail-in rebate), sponsors (e.g., waiver of co-payment when an off-brand alternative is selected), and pharmacies (e.g., in-store coupon). The EPP server may broadcast the bid responses to the pharmacies and receive update bid responses. When the auction closes, the EPP server sends to the EPP application pharmacy options based on the bid responses.

As described above, the EPP system provides a registration service that allows patients to register to effectively activate their membership with the prescription benefit plan. The EPP system may require a patient to activate their membership before the patient can take advantage of the prescription benefit plan (e.g., provided by the patient's employer). To active their membership, a patient uses a computer (e.g., smartphone) to interact with a web site (or other service) provided by the EPP system. The activation process may require that the patient supply contact information such as a cell phone number or email address so that messages can be sent to the patient. The activation process may also allow a patient to download the EPP client to the patient's computer. When a sponsor registers a patient, the sponsor may also provide contact information of patients so that the EPP system can remind the patients to activate their membership.

In some embodiments, the EPP server may provide a user experience for selecting prescription options when the patient is not visiting with the prescriber and does not have an EPP client. To provide the user experience, the EPP server receives from a pharmacy a notification of a prescription, which includes a prescribed drug, a quantity, and a dosage strength. The EPP server may provide an application programming interface that allows a pharmacy to submit such notifications. As discussed above, the EPP server identifies prescription options relating to the prescribed drug. The prescription options may relate to a substitute drug that does not need approval by the prescriber, an alternative drug that needs approval by the prescriber, an alternative pharmacy for dispensing the prescribed drug, splitting a larger dosage strength (e.g., 25.0 mg) of the prescribed drug into a smaller dosage strength (e.g., 12.5 mg) specified by the prescription, a longer supply (e.g., 90 days) of the prescribed drug than a smaller supply (e.g., 30 days) specified by the prescription, and so on. The EPP server may provide to the patient a reason for each prescription option such as the substitute drug is a lower-cost, generic version of the prescribed drug, the prescribed drug is not approved under the prescription benefit plan but the alternative drug is, the price of the alternative pharmacy is less than that of the pharmacy, and so on.

In some embodiments, the EPP server may send an electronic message (e.g., text message or email message) to a patient device that includes a link to a web page that identifies the various prescription options. The patient can review the prescription options and select one or more of the prescription options. For example, the patient may select multiple prescription options such as an alternative pharmacy, a substitute drug, and a larger supply. Alternatively, the EPP server may provide an application programming interface through which an EPP client can retrieve prescription options and display the prescription options for review by the patient.

When a prescription option is selected, the EPP server provides a notification to the patient or a pharmacy based on the selected prescription option. When the selected prescription option relates to dispensing the prescription by a pharmacy to which the prescription was sent, the EPP server sends instructions to the pharmacy to proceed with dispensing the prescription. The EPP server may only provide this prescription option when the prescribed drug is an approved drug under the patient's prescription benefit plan.

When the selected prescription option relates to a substitute drug that does not need approval by the prescriber, the EPP server sends instructions to the pharmacy to dispense the substitute drug. Alternatively, or additionally, the EPP server may send instructions to the prescriber to request the substitute drug from the pharmacy.

When the selected prescription option relates to an alternative drug that needs approval by the prescriber, the EPP server sends instructions to the patient device to inform the patient to contact the prescriber for a new prescription for the alternative drug. The instructions to contact the prescriber may include a link for electronically contacting the prescriber, for example, using a secure messaging system of the prescriber. Alternatively, or additionally, the EPP server may send instructions to the prescriber.

When the selected prescription option relates to an alternative pharmacy for dispensing the prescribed drug, the EPP server sends the instructions to the patient and provides guidance on transferring the prescription to the alternative pharmacy. Alternatively, or additionally, the EPP server may send instructions to the pharmacy to transfer the prescription to the alternative pharmacy. Also, the EPP server may send instructions to alternative pharmacy informing them to request the pharmacy to transfer the prescription.

When the selected prescription option relates to splitting a larger dosage strength of the prescribed drug into a smaller dosage strength specified by the prescription, the EPP server sends instructions to the patient to inform the patient to request the prescribed drug in the larger dosage strength. Alternatively, or additionally, the EPP server may send the instructions to the pharmacy to dispense the prescribed drug in the larger dosage strength.

When the selected prescription option relates to a larger supply of the prescribed drug than a smaller supply specified by the prescription, the EPP server sends instructions to the patient to inform the patient to request the larger supply of the prescribed drug. Alternatively, or additionally, the EPP server may send instructions to the pharmacy to dispense the larger supply of the prescribed drug.

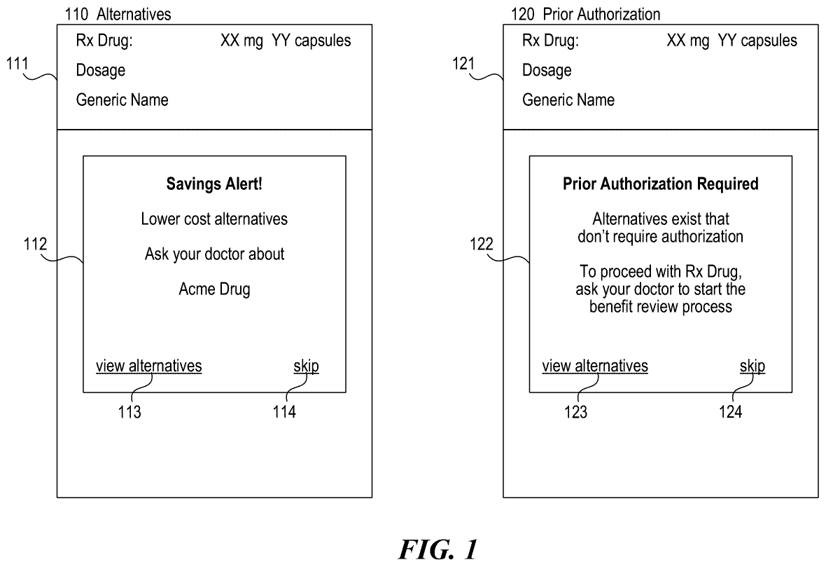

FIGS. 1-3 illustrate a user experience for a patient that is provided by the EPP system in some embodiments. Referring to FIG. 1, display 110 displays alternative information that includes a prescription area 111 and an alert area 112. The prescription area identifies the prescribed drug, the dosage and quantity, the generic name of the drug, refill information, and so on. The alert area indicates that there is a lower-cost drug alternative. The alert area also includes a view alternatives link 113 and a skip link 114. The patient can select the view alternatives link to view the drug options for the alternatives. The drug options may list the name of the drug, per-pill amount (e.g., 5 mg), manufacturer or sponsor rebates, and so on. The patient can then select a drug option. Display 120 displays authorization information that includes a prescription area 121 and an authorization area 122. The authorization area indicates that the prior authorization is required for the selected drug option. The authorization area also includes a review alternatives link 123 and a done link 124. The patient can select the view alternatives link to re-display the drug options and the done button to proceed with submitting a request for prior authorization.

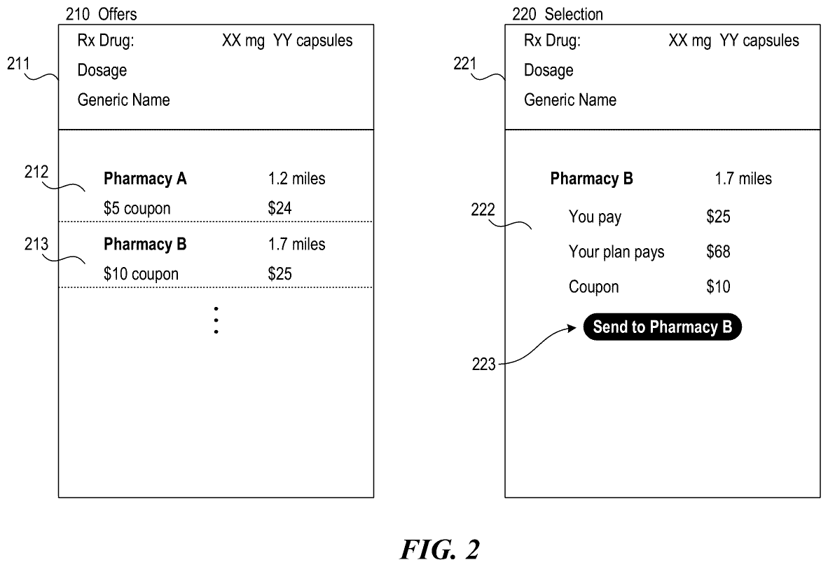

Referring to FIG. 2, display 210 displays pharmacy information for multiple pharmacy options that includes a prescription area 211 and pharmacy options 212-213. The pharmacy options may identify the pharmacy (e.g., Pharmacy A), the current or other distance to the pharmacy (e.g., 1.2 miles), the patient price (e.g., $24), incentive information (e.g., $5 coupon), and so on. Display 220 displays pharmacy information for a single pharmacy that includes a prescription area 221, detailed pharmacy information 222 for the selected pharmacy, and a send to pharmacy or pay now link 223. The detailed pharmacy information may contain more detailed information than is illustrated by the pharmacy options of display 210. The patient can then select the send to pharmacy link to submit the prescription to be dispensed by the selected pharmacy.

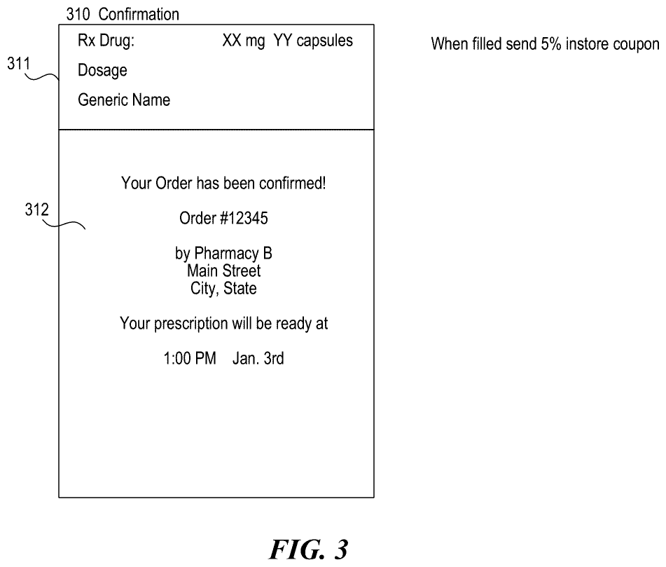

Referring to FIG. 3, display 310 displays a confirmation received from the pharmacy via the EPP server that includes a prescription area 311 and a confirmation area 312. The confirmation area may provide an order number, the patient price, the pharmacy address, and an indication of when the pharmacy will have the prescription ready. Although not illustrated, when a patient picks up a prescription from a pharmacy, the pharmacy may forward to the EPP server an indication of a coupon to be provided to the patient. The EPP server then forwards an indication of the component such as a QR code so that the patient can redeem the coupon after the prescription has been picked up. A QR code may also be provided that a patient can scan at a pharmacy to confirm authorization to pick up a prescription. For example, a patient may provide payment information at the time of confirmation and use the QR code to confirm authorization to pick up the prescription from a kiosk.

In some embodiments, the EPP client may include a cancel button to allow a patient to withdraw a prescription from the pharmacy to which it was sent. Upon receiving notification of the withdrawal, the EPP server may contact the pharmacy system to ensure that the prescription has not been picked up. Also, when a patient picks up a prescription, the pharmacy system may notify the EPP system. When a patient withdraws a prescription, the EPP server may then collect new pharmacy options (including conducting an auction) so that the patient can select a new pharmacy based on the then-current pharmacy options. Similarly, when a patient is to order a refill, the EPP server may present then-current pharmacy options. The EPP server can coordinate the transfer of the prescription.

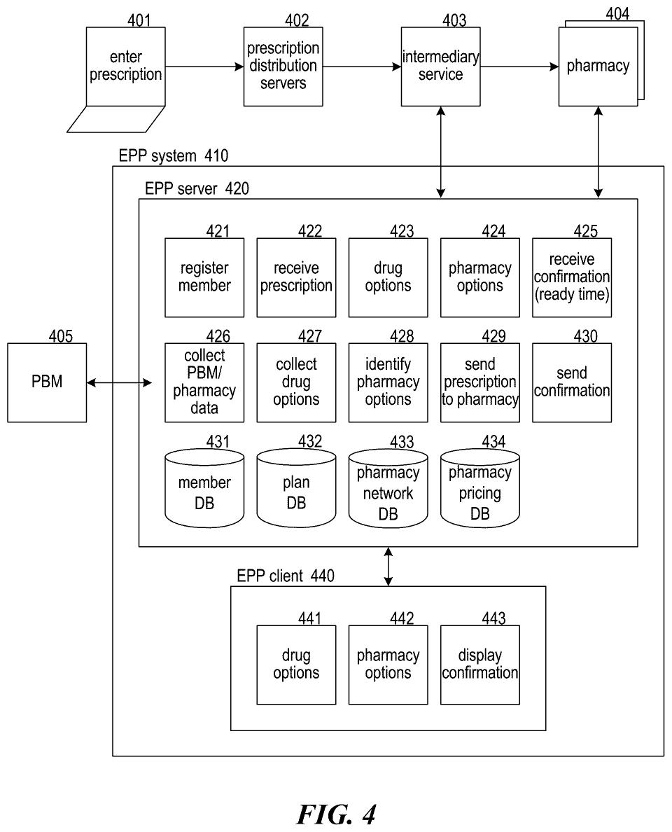

FIG. 4 is a block diagram that illustrates components of the EPP system in some embodiments. A prescriber system 401 may communicate with a prescription routing service 402 to submit prescriptions to be distributed to the pharmacies. The prescription routing service may communicate with intermediary service 403 to forward prescriptions to be processed by the EPP system. The intermediary service may communicate with EPP system 410 to send prescriptions and with pharmacies 404 to transfer or forward prescriptions. The EPP system may be connected to PBM systems 405 to receive benefit plan information such as formulary information, identification of a plan member, contact information (e.g., e.g. cell phone number) for a plan member, and so on. The EPP system may include a server 420 and EPP client 440. The EPP server includes a registered member component 421 that controls the registration of patients, for example, in response to receiving information on those covered under a plan from a PBM system. The registered patients are referred to as members. The register member component may store membership information in the member database 431. The EPP server also includes a receive prescription component 422 that receives prescriptions from the intermediary service. The EPP system may also include a drug options component 423 that retrieves drug option information from a plan database 432 and other databases such as one that provides detailed information about alternative drugs. The drug options component then sends the drug options to the EPP client as described above. The EPP system may include a pharmacy option component 424 that coordinates the selection of a pharmacy option. The pharmacy option component is provided a prescribed drug and retrieves information on the pharmacies that are authorized by a plan from pharmacy network database 433 and pricing information from pharmacy pricing database 434. The pharmacy options component receives from an EPP client a selection of a pharmacy and forwards the prescription to the selected pharmacy. The pharmacy options component may implement an auction to determine current pricing information for each pharmacy. The EPP system includes a receive confirmation component 425 that receives confirmation from the selected pharmacy and forwards the confirmation to the EPP client.

The EPP system includes a collect PBM/pharmacy data component 426 that collects pharmacy and PBM data. The EPP system includes a collect drug options component 427 that collects information on drug options, such as alternatives to various drugs, for a prescription. The EPP system includes an identify pharmacies options component 428 that identifies pharmacy options for dispensing a prescription. The EPP system includes a send prescription component 429 that sends a prescription to a pharmacy. The prescription may be sent directly to the pharmacy 404 or to an intermediary service 403 that sends the prescription to the pharmacy. The EPP system includes a send confirmation component 430 that controls the sending of confirmations based on information stored in the member database for the patient.

The EPP client includes a drug options component 441, a pharmacy options component 442, and a display confirmation component 443. The drug options component receives drug options from the EPP server (and may receive incentive information from manufacturers and plan sponsors) and displays the drug options (and any incentive information). The pharmacy options component receives pharmacy options (which may include pharmacy incentives) from the EPP server, displays the pharmacy options, receives a selection of the pharmacy option, and forwards an indication of the selected pharmacy option to the EPP server. The display confirmation component receives an indication of a pharmacy confirmation from the EPP server and displays the confirmation.

Although not illustrated, the EPP client may also include a pre-authorization component and an equivalent options component. When a plan sponsor requires pre-authorization for a drug, the EPP server may notify the EPP client. The pre-authorization component may provide the pre-authorization information to the patient and request any information needed to submit the pre-authorization. The pre-authorization component then sends the request for pre-authorization to the EPP server, receives the pre-authorization response, and provides the response to the patient. If pre-authorization is denied, the pre-authorization component may coordinate submitting of an appeal request or selecting of an alternative drug and notifying the prescriber so that a new prescription can be written. The equivalent options component may help coordinate the selection of pharmacologically equivalent drugs (e.g., a generic drug) for a prescribed drug (e.g., a branded drug).

The computing systems (e.g., network nodes or collections of network nodes) on which the EPP system may be implemented may include a central processing unit, input devices, output devices (e.g., display devices and speakers), storage devices (e.g., memory and disk drives), network interfaces, graphics processing units, cellular radio link interfaces, global positioning system devices, and so on. The input devices may include keyboards, pointing devices, touch screens, gesture recognition devices (e.g., for air gestures), head and eye tracking devices, microphones for voice recognition, and so on. The computing systems may include desktop computers, laptops, tablets, e-readers, personal digital assistants, smartphones, gaming devices, servers, and so on. The computing systems may access computer-readable media that include computer-readable storage media and data transmission media. The computer-readable storage media are tangible storage means that do not include a transitory, propagating signal. Examples of computer-readable storage media include memory such as primary memory, cache memory, and secondary memory (e.g., DVD) and other storage. The computer-readable storage media may have recorded on them or may be encoded with computer-executable instructions or logic that implements the EPP system. The data transmission media are used for transmitting data via transitory, propagating signals or carrier waves (e.g., electromagnetism) via a wired or wireless connection. The computing systems may include a secure cryptoprocessor as part of a central processing unit for generating and securely storing keys and for encrypting and decrypting data using the keys.

The EPP system may be described in the general context of computer-executable instructions, such as program modules and components, executed by one or more computers, processors, or other devices. Generally, program modules or components include routines, programs, objects, data structures, and so on that perform tasks or implement data types of the EPP system. Typically, the functionality of the program modules may be combined or distributed as desired in various examples. Aspects of the EPP system may be implemented in hardware using, for example, an application-specific integrated circuit (“ASIC”) or field programmable gate array (“FPGA”).

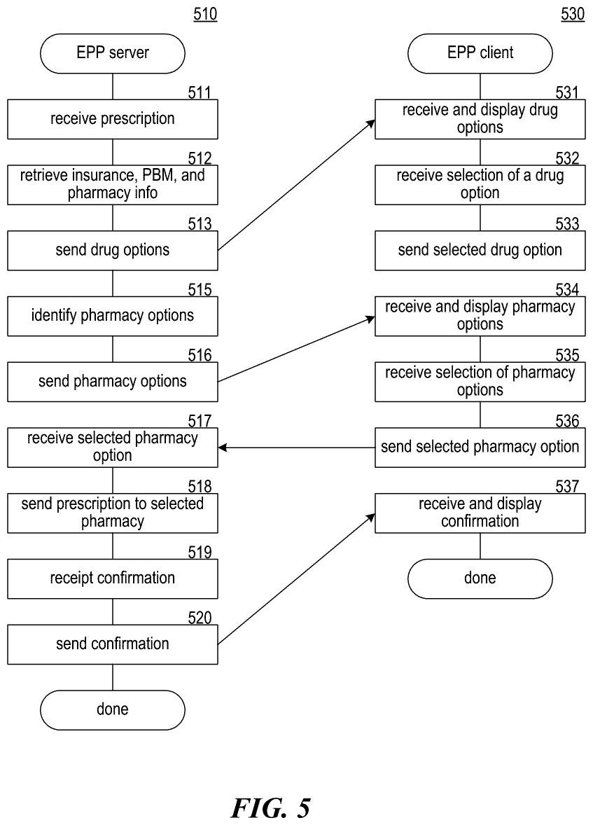

FIG. 5 includes flow diagrams that illustrate the processing of an EPP server and EPP client in some embodiments. A flow diagram 510 illustrates the processing of an EPP server, and a flow diagram 530 illustrates the processing of an EPP client. In block 511, the EPP server receives a prescription from a prescriber system or an intermediary service. In block 512, the server retrieves prescription benefit plan, PBM, pharmacy information, and so on associated with the prescription. In block 513, the EPP server sends indication of the drug options to the EPP client. In block 531, upon receiving the drug options, the EPP client displays the drug options. The patient may then coordinate the selection of new drug with the prescriber. Alternatively, the EPP system may communicate with the prescriber to write a new prescription. In block 532, the EPP client receives a selection of a drug option. In block 533, the EPP client sends the selected drug option to the prescriber so that the prescriber can the write a new prescription and send it to the EPP server. If a new prescription is written, then when the EPP server receives the new prescription in block 511. In block 515, when the EPP server determines the prescription is not to be replaced, the EPP server identifies pharmacy options based on the prescribed drug. In block 516, the EPP server sends an indication of the pharmacy options to the EPP client. In block 534, upon receiving the pharmacy options, the EPP client displays the pharmacy options. In block 533, the EPP client receives a selection of the pharmacy option. In block 536, the EPP client sends an indication of the selected pharmacy option to the server. In block 517, the EPP server receives the selected pharmacy option. In block 518, the EPP server sends the prescription to the selected pharmacy. In block 519, the EPP server receives the confirmation from the selected pharmacy. In block 520, the EPP server sends a confirmation to the EPP client and then completes. In block 537, upon receiving indication of the confirmation, the EPP client displays the confirmation and then completes.

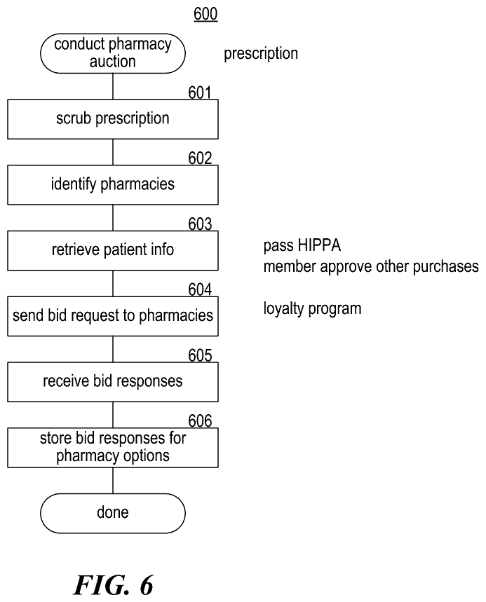

FIG. 6 is a flow diagram that illustrates the conducting of a pharmacy auction. A conduct pharmacy option component 600 receives a prescription and conducts an auction to determine patient prices along with incentives provided by pharmacies. In block 601, the component may scrub the prescription to ensure that any information forwarded to pharmacy complies with the Health Insurance Portability and Accountability Act (“HIPAA”) of the United States. In block 602, the component identifies pharmacies to participate in the auction. In block 603, the component may retrieve patient information in compliance with HIPAA requirements. In block 604, the component sends a bid request to each pharmacy. In block 605, the component receives bid responses. The component may also publicize each bid to allow pharmacies to revise their prior bids. In block 606, the component stores the bid responses for use when providing pharmacy options and then completes.

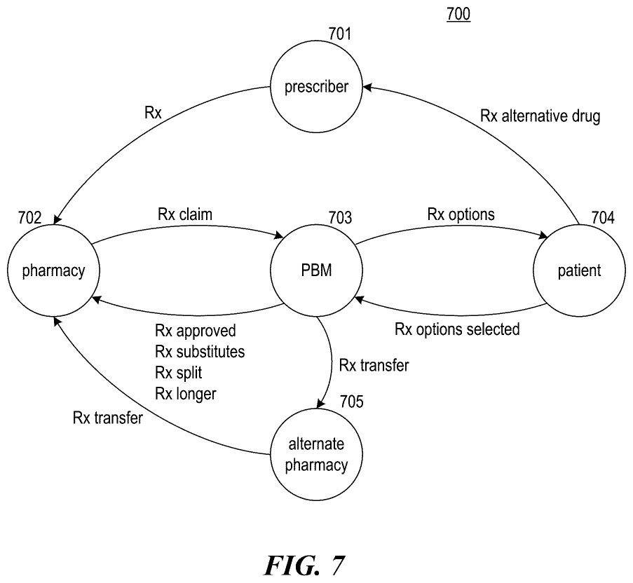

FIG. 7 is a diagram that illustrates the flow of data relating to prescription options. Initially, a prescriber 701 sends a prescription for a patient to a pharmacy. Upon receiving the prescription, the pharmacy 702 processes the prescription and forwards to a PBM. The PBM 703 checks whether the prescription is covered by the prescription benefit plan of the patient and identifies prescription options. The PBM then sends the prescription options to the patient. The patient 704 can review the prescription options and send to the PBM an indication of the selection prescription option. Also, if the patient selects an alternative drug, the patient can request the prescriber to issue a new prescription for the alternative drug. Upon receiving the selection of the prescription options, the PBM notifies the pharmacy and may notify the alternative pharmacy of the selection drug option. When the alternative pharmacy 705 receives notification of the alternative pharmacy that has been selected as the dispensing pharmacy, the alternative pharmacy may notify the pharmacy to transfer the prescription.

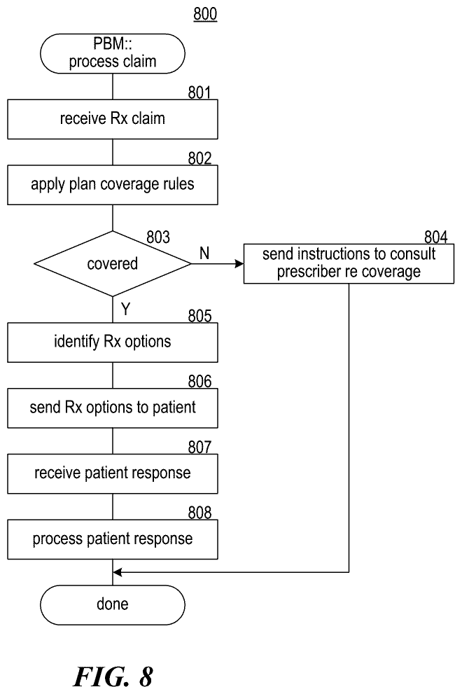

FIG. 8 is a flow diagram that illustrates the processing of a claim by a pharmacy benefit manager system in some embodiments. The process claim component 800 of a PBM system identifies prescription options and processes the prescription option selected by a patient. In block 801, the component receives a prescription claim from a pharmacy. In block 802, the component applies rules of the prescription benefit plan to the prescription. In decision block 803, if the prescription is covered, then the component continues at block 805, else the component continues at block 804. In block 804, the component sends instructions to the patient to consult the prescriber to discuss options and then completes. Although not illustrated, the component may also provide various prescription options to the patient when a prescription is not covered. In block 805, the component identifies the various prescription options that are available. In block 806, the component sends the prescription options to the patient. In block 807, the component receives the patient response, which may identify one or more prescription options. In block 808, the component invokes a process patient response component to proceed with the selected prescription options. Although not illustrated, the component may invoke the process patient response component to process each prescription option selected by the patient. The component then completes.

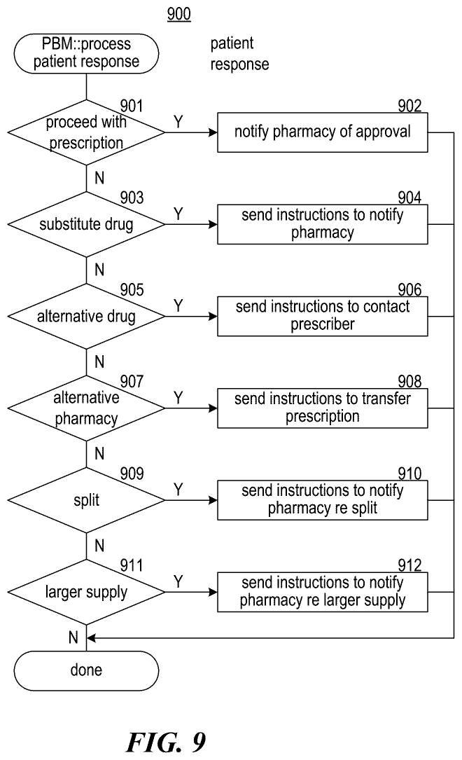

FIG. 9 is a flow diagram that illustrates processing of a process patient response component of a pharmacy benefit manager in some embodiments. The process patient response component 900 receives a patient response that identifies a prescription option such as proceed with the prescription, proceed with an alternative pharmacy, and so forth. In decision block 901, if selected prescription option is to proceed with the prescription, then the component continues at block 902, else the component continues at block 903. In block 902, the component notifies the pharmacy of the approval of the prescription and then completes. In decision block 903, if the selected prescription option is for a substitute drug, then the component continues at block 904, else the component continues at block 905. In block 904, the component sends instructions to notify the pharmacy of the substitute drug and then completes. In decision block 905, if the selected prescription option is for an alternative drug, then the component continues at block 906, else the component continues at block 907. In block 906, the component sends instructions to the patient to contact the prescriber to request a prescription for the alternative drug and then completes. In decision block 903, if the selected option is for an alternative pharmacy, then the component continues at block 908, else the component continues at block 909. In block 909, the component sends instructions to transfer the prescription to the alternate pharmacy and then completes. The instructions may be sent to the original pharmacy, the alternate pharmacy, or the prescriber. In decision block 909, if the selected prescription option is to split a larger dosage strength, the component continues at block 910, else the component continues at block 911. In block 910, the component sends instructions to notice the pharmacy of the split and then completes. In decision block 911, if the selected option is for a larger supply of the prescribed drug, then the component continues at block 912, else the component completes. In block 912, the component sends instructions to notify the pharmacy to dispense the larger supply and then completes.

The following paragraphs describe various embodiments of aspects of the EPP system. An implementation of the EPP system may employ any combination of the embodiments.

In some embodiments, a method performed by a computing system is provided for providing to a patient a user experience for securely submitting a prescription to a pharmacy. The method receives a notification of a prescription prescribed by a prescriber for the patient. The prescription specifies a prescribed drug for the patient. The notification is provided by a prescription intermediary that receives the prescription electronically from the prescriber. The method displays to the patient information on drug options relating to the prescribed drug. The method displays to the patient pharmacy options relating to the selected drug option. Each pharmacy option identifies a pharmacy. The method receives from the patient a selection of a pharmacy option. The method directs that an indication of the prescription be sent to the pharmacy identified by the selected pharmacy option so that that pharmacy can fill the prescription. In some embodiments, the drug options are in accordance with a prescription benefit plan through which the patient has prescription coverage. In some embodiments, prescription intermediary is a service that receives the prescription directly from the prescriber. In some embodiments, the prescription intermediary is a service that receives the prescription via a prescription switch that receives the prescription from the prescriber and routes the prescription to the prescription intermediary. In some embodiments, the prescription intermediary is a service controlled by an intermediary pharmacy that transfers the prescription to the pharmacy identified by the selected pharmacy option. In some embodiments, a pharmacy option identifies pricing information and an incentive provided by the pharmacy identified by the pharmacy option. In some embodiments, the computing system is a smartphone of the patient. In some embodiments, the receiving of the notification and the directing are performed in real-time so that the patient can discuss the drug options and pharmacy options with the prescriber when the prescription is prescribed by the prescriber.

In some embodiments, a computing system is provided for coordinating submitting of a prescription prescribed by a prescriber to a pharmacy. The computing system comprises one or more computer-readable storage mediums for storing computer-executable instructions and one or more processors for executing the computer-executable instructions stored in the one or more computer-readable storage mediums. The instructions control the computing system to receive a prescription for a drug prescribed by a prescriber for a patient. The instructions control the computing system to send to the patient information on pharmacies for filling the prescription. The instructions control the computing system to receive from the patient a selection of a pharmacy. The instructions control the computing system to send the prescription to the selected pharmacy so that that selected pharmacy can fill the prescription. In some embodiments, the instructions further control the computing system to store in a blockchain data structure prescription transaction identifying the prescription and a pharmacy transaction identifying the pharmacy and the prescription. In some embodiments, the instructions further control the computing system to display to the patient information on drug options relating to the prescribed drug and receive from the patient a selection of a drug option wherein the information on pharmacies is based on the selected drug option. In some embodiments, the instructions further control the computing system to access information relating to a prescription benefit plan through which the patient has prescription coverage. In some embodiments, a pharmacy option identifies pricing information and an incentive provided by the pharmacy identified by the pharmacy option. In some embodiments, the computer-executable instructions control the computing to retrieve information from a pharmacy benefit manager. In some embodiments, the computer-executable instructions control the computing to verify patient eligibility and pricing with the pharmacy benefit manager. In some embodiments, the prescription is received from an intermediary service that receives prescriptions from prescribers and sends the prescriptions to pharmacies indicated by the prescriptions. In some embodiments, the computing system is associated with a pharmacy that is registered with the intermediary service. In some embodiments, the patient information is sent to and the selection of a pharmacy is received from a smartphone of the patient.

In some embodiments, a method performed by a computing system of a patient is provided for submitting a prescription to a pharmacy. The method receives a notification of a prescription prescribed by a prescriber for the patient. The prescription specifies a prescribed drug for the patient. The notification is sent by a service that receives prescription from prescribers and that routes the prescriptions to pharmacies identified by the prescribers. The method displays to the patient pharmacy options relating to the prescribed drug. Each pharmacy option identifies a pharmacy and provides pricing information for that pharmacy. The method receives from the patient a selection of a pharmacy option. The method directs that an indication of the prescription be sent to the pharmacy identified by the selected pharmacy option so that that pharmacy can fill the prescription. In some embodiments, the pharmacy options are in accordance with a prescription benefit plan through which the patient has prescription drug coverage.

In some embodiments, a method performed one or more computing systems is provided that provides prescription options relating to a prescription prescribed by a prescriber for a patient. The method receives receiving from a pharmacy system a notification of the prescription that specifies a prescribed drug for the patient. The method identifies identifying prescription options relating to the prescribed drug. The method sends to a patient device of the patient an indication of the prescription options. The method receives from the patient device a selection by the patient of a prescription option. The method sends instructions relating to proceeding with the selected prescription option. In some embodiments, the drug options are in accordance with a prescription benefit plan through which the patient has prescription coverage. In some embodiments, the patient device is a smartphone of the patient. In some embodiments, the selected prescription option relates to a substitute drug that does not need approval by the prescriber and wherein the instructions are sent to the pharmacy system to inform the pharmacy of the substitute drug. In some embodiments, the selected prescription option relates to an alternative drug that needs approval by the prescriber and wherein the instructions are sent to the patient device to inform the patient to contact the prescriber for a new prescription for the alternative drug. In some embodiments, the selected prescription option relates to an alternative pharmacy for dispensing the prescribed drug and wherein the instructions are sent to the patient device to inform the patient to transfer the prescription to the alternative pharmacy. In some embodiments, the selected prescription option relates to splitting a larger dosage strength of the prescribed drug into a smaller dosage strength specified by the prescription and wherein the instructions are sent to the patient device to inform the patient to request the prescribed drug in the larger dosage strength. In some embodiments, the selected prescription option relates to a longer supply of the prescribed drug than a smaller supply specified by the prescription and wherein the instructions are sent to the patient device to inform the patient to request the larger supply of the prescribed drug.

In some embodiments, one or more computing systems is provided for providing prescription options relating to a prescription prescribed by a prescriber for a patient. The one or more computing systems includes one or more computer-readable storage mediums for storing computer-executable instructions and one or more processors for executing the computer-executable instructions stored in the one or more computer-readable storage mediums. The instructions control the one or more computing systems to receive a notification of the prescription, the prescription having a prescribed drug, a quantity, and a dosage strength. The instructions control the one or more computing systems to identify prescription options relating to the prescribed drug. The instructions control the one or more computing systems to send to a patient device of the patient an indication of the prescription options. The instructions control the one or more computing systems to receive from the patient device a selection by the patient of a prescription option. The instructions control the one or more computing systems to send instructions relating to proceeding with the selected prescription option.

In some embodiments, the one or more computing systems of claim 9 the selected prescription option relates to dispensing the prescription by a pharmacy to which the prescription was sent and wherein the instructions are sent to the pharmacy to proceed with dispensing the prescription. In some embodiments, the selected prescription option relates to a substitute drug that does not need approval by the prescriber and wherein the instructions are sent to a pharmacy from which the notification of the prescription was received to inform the pharmacy of the substitute drug. In some embodiments, the selected prescription option relates to an alternative drug that needs approval by the prescriber and wherein the instructions are sent to the patient device to inform the patient to contact the prescriber for a new prescription for the alternative drug. In some embodiments, the instructions include a link for electronically contacting the prescriber. In some embodiments, the link relates to a secure messaging system of the prescriber. In some embodiments, the selected prescription option relates to an alternative pharmacy for dispensing the prescribed drug and wherein the instructions are sent to the patient device and provide guidance on transferring the prescription to the alternative pharmacy. In some embodiments, the selected prescription option relates to an alternative pharmacy for dispensing the prescribed drug and wherein the instructions are sent to a pharmacy from which the notification of the prescription was received to inform the pharmacy to transfer the prescription to the alternative pharmacy. In some embodiments, the selected prescription option relates to an alternative pharmacy for dispensing the prescribed drug and wherein the instructions are sent to the alternative pharmacy and indicate to request a pharmacy from which the notification of the prescription was received to transfer the prescription to the alternative pharmacy. In some embodiments, the selected prescription option relates to splitting a larger dosage strength of the prescribed drug into a smaller dosage strength specified by the prescription and wherein the instructions are sent to the patient device to inform the patient to request the prescribed drug in the larger dosage strength. In some embodiments, the selected prescription option relates to splitting a larger dosage strength of the prescribed drug into a smaller dosage strength specified by the prescription and the instructions are sent a pharmacy from which the notification of the prescription was received to inform the pharmacy to dispense the prescribed drug in the larger dosage strength. In some embodiments, the selected prescription option relates to a longer supply of the prescribed drug than a smaller supply specified by the prescription and wherein the instructions are sent to the patient device to inform the patient to request the larger supply of the prescribed drug. In some embodiments, the selected prescription option relates to a longer supply of the prescribed drug than a smaller supply specified by the prescription and the instructions are sent to a pharmacy from which the notification of the prescription was received to inform the pharmacy to dispense the larger supply of the prescribed drug.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims. Accordingly, the invention is not limited except as by the appended claims.

I/We claim:

1. A method performed one or more computing systems for providing prescription options relating to a prescription prescribed by a prescriber for a patient, the method comprising:
receiving from a pharmacy system a notification of the prescription that specifies a prescribed drug for the patient;
identifying prescription options relating to the prescribed drug;
sending to a patient device of the patient an indication of the prescription options;
receiving from the patient device a selection by the patient of a prescription option; and
sending instructions relating to proceeding with the selected prescription option.

2. The method of claim 1 wherein the drug options are in accordance with a prescription benefit plan through which the patient has prescription coverage.

3. The method of claim 1 wherein the patient device is a smartphone of the patient.

4. The method of claim 1 wherein the selected prescription option relates to a substitute drug that does not need approval by the prescriber and wherein the instructions are sent to the pharmacy system to inform the pharmacy of the substitute drug.

5. The method of claim 1 wherein the selected prescription option relates to an alternative drug that needs approval by the prescriber and wherein the instructions are sent to the patient device to inform the patient to contact the prescriber for a new prescription for the alternative drug.

6. The method of claim 1 wherein the selected prescription option relates to an alternative pharmacy for dispensing the prescribed drug and wherein the instructions are sent to the patient device to inform the patient to transfer the prescription to the alternative pharmacy.

7. The method of claim 1 wherein the selected prescription option relates to splitting a larger dosage strength of the prescribed drug into a smaller dosage strength specified by the prescription and wherein the instructions are sent to the patient device to inform the patient to request the prescribed drug in the larger dosage strength.

8. The method of claim 1 wherein the selected prescription option relates to a longer supply of the prescribed drug than a smaller supply specified by the prescription and wherein the instructions are sent to the patient device to inform the patient to request the larger supply of the prescribed drug.

9. One or more computing systems for providing prescription options relating to a prescription prescribed by a prescriber for a patient, the one or more computing systems comprising:
one or more computer-readable storage mediums for storing computer-executable instructions for controlling the one or more computing systems to:
receive a notification of the prescription, the prescription having a prescribed drug, a quantity, and a dosage strength;
identify prescription options relating to the prescribed drug;
send to a patient device of the patient an indication of the prescription options;
receive from the patient device a selection by the patient of a prescription option; and
send instructions relating to proceeding with the selected prescription option; and
one or more processors for executing the computer-executable instructions stored in the one or more computer-readable storage mediums.

10. The one or more computing systems of claim 9 wherein the selected prescription option relates to dispensing the prescription by a pharmacy to which the prescription was sent and wherein the instructions are sent to the pharmacy to proceed with dispensing the prescription.

11. The one or more computing systems of claim 9 wherein the selected prescription option relates to a substitute drug that does not need approval by the prescriber and wherein the instructions are sent to a pharmacy from which the notification of the prescription was received to inform the pharmacy of the substitute drug.

12. The one or more computing systems of claim 9 wherein the selected prescription option relates to an alternative drug that needs approval by the prescriber and wherein the instructions are sent to the patient device to inform the patient to contact the prescriber for a new prescription for the alternative drug.

13. The one or more computing systems of claim 12 wherein the instructions include a link for electronically contacting the prescriber.

14. The one or more computing systems of claim 13 wherein the link relates to a secure messaging system of the prescriber.

15. The one or more computing systems of claim 9 wherein the selected prescription option relates to an alternative pharmacy for dispensing the prescribed drug and wherein the instructions are sent to the patient device and provide guidance on transferring the prescription to the alternative pharmacy.

16. The one or more computing systems of claim 9 wherein the selected prescription option relates to an alternative pharmacy for dispensing the prescribed drug and wherein the instructions are sent to a pharmacy from which the notification of the prescription was received to inform the pharmacy to transfer the prescription to the alternative pharmacy.

17. The one or more computing systems of claim 9 wherein the selected prescription option relates to an alternative pharmacy for dispensing the prescribed drug and wherein the instructions are sent to the alternative pharmacy and indicate to request a pharmacy from which the notification of the prescription was received to transfer the prescription to the alternative pharmacy.

18. The one or more computing systems of claim 9 wherein the selected prescription option relates to splitting a larger dosage strength of the prescribed drug into a smaller dosage strength specified by the prescription and wherein the instructions are sent to the patient device to inform the patient to request the prescribed drug in the larger dosage strength.

19. The one or more computing systems of claim 9 wherein the selected prescription option relates to splitting a larger dosage strength of the prescribed drug into a smaller dosage strength specified by the prescription and wherein the instructions are sent a pharmacy from which the notification of the prescription was received to inform the pharmacy to dispense the prescribed drug in the larger dosage strength.

20. The one or more computing systems of claim 9 wherein the selected prescription option relates to a longer supply of the prescribed drug than a smaller supply specified by the prescription and wherein the instructions are sent to the patient device to inform the patient to request the larger supply of the prescribed drug.

21. The one or more computing systems of claim 9 wherein the selected prescription option relates to a longer supply of the prescribed drug than a smaller supply specified by the prescription and wherein the instructions are sent to a pharmacy from which the notification of the prescription was received to inform the pharmacy to dispense the larger supply of the prescribed drug.
